(19) United States
(12) Patent Application Publication
(10) Pub. No.: US 2024/0325415 A1
(43) Pub. Date: Oct. 3, 2024

(54) COMPOSITIONS COMPRISING A CONSTITUENT, DERIVATIVE OR EXTRACT OF CANNABIS

(71) Applicant: Nicoventures Trading Limited, London (GB)

(72) Inventors: Steven L ALDERMAN, Winston-Salem, NC (US); Thomas POOLE, Winston-Salem, NC (US); Karen TALUSKIE, Winston-Salem, NC (US); Kathryn L WILBERDING, Winston-Salem, NC (US); Jenni HAWKE, London (GB); Ashley DAVIES, London (GB); Michael S DANIEL, Winston-Salem, NC (US); Kai TANG, Winston-Salem, NC (US); Keyi XU, Winston-Salem, NC (US); Karina MCQUILLAN, London (GB); John CARAWAY, Winston-Salem, NC (US)

(21) Appl. No.: 18/580,005

(22) PCT Filed: Jul. 21, 2022
(86) PCT No.: PCT/GB2022/051903

§ 371 Date: Jan. 17, 2024

Related U.S. Application Data
(60) Provisional application No. 63/224,589, filed on Jul. 22, 2021.

(51) Int. Cl. A61K 31/00; A61K 9/00; A61K 45/06; A61K 47/14; A61K 47/32; A61K 47/38; A61K 47/40

(52) U.S. Cl. CPC A61K 31/658; A61K 9/006; A61K 36/3482; A61K 45/06; A61K 47/14; A61K 47/32; A61K 47/38; A61K 47/40

(57) ABSTRACT

The present invention relates to compositions comprising active agents formulated for both buccal and gastric delivery wherein the composition also comprises one or more constituent, derivative or extract of cannabis and a further physiologically active agent.

### TECHNICAL FIELD

The present invention relates to compositions comprising active agents formulated for both buccal and gastric delivery wherein the composition also comprises one or more constituent, derivative or extract of cannabis and a further physiologically active agent.

### BACKGROUND

Cannabinoids are compounds derived from Cannabis sativa, an annual plant in the Cannabaceae family. The plant contains about 60 cannabinoids.

Cannabinoid molecules are known to cause a variety of therapeutic and psychotropic effects when administered to a subject. In particular, the use of one specific cannabinoid, delta-9 tetrahydrocannabinol (THC), causes a mild temporary psychotropic effect in users. However, it is well known that dozens of other cannabinoids are also present in cannabis, none of which have psychotropic effects, and which have, or potentially may have, beneficial pharmacological effects in humans. These alternate cannabinoids which are devoid of psychotropic effect include but are not limited to tetrahydrocannabinolic acid (THCA), cannabinolic acid (CBNA), cannabidiolic acid (CBDA) and cannabigerolic acid (CBGA) and the de-carboxylated derivatives cannabinol (CBN), cannabidiol (CBD) and cannabigerol (CBG).

Cannabinoids are key players in biological systems and bind to receptors in the body known as cannabinoid receptors which have been implicated in a variety of physiological functions, including appetite, pain, emotional behaviour (mood), memory, and inflammation. There are currently two known well defined subtypes of cannabinoid receptors, called CB1 and CB2, both of which are G protein-coupled receptors. The CB1 receptor (CB1R) is expressed mainly in the brain (central nervous system or “CNS”), and also in the lungs, liver and kidneys. The CB2 receptor (CB2R) is expressed mainly in the immune system and in hematopoietic or blood cells.

In general, cannabinoid molecules have traditionally been administered via inhalation, for example by smoking or heating and inhaling the vapours of cannabis plants or cannabis extracts. Oral administration of such molecules, as compared with administration via inhalation, can provide improved consistency of dosing, more discreet administration, and increased convenience for users.

Cannabinoids are lipophilic and potentially acid-labile compounds. Because of their hydrophobic nature, cannabinoids are poorly absorbed systemically from oral dosage forms because of the poor dissolution of cannabinoids in the aqueous environment of gastrointestinal tract. Oral formulations of cannabinoids, therefore, exhibit low bioavailability. The present invention combines different delivery forms to maximise bioavailability as well as to deliver an additional physiological active.

### SUMMARY

According to a first embodiment described herein, there is provided a composition comprising a first active agent formulated for buccal delivery and a second active agent formulated for gastric delivery, the composition comprising one or more constituent, derivative or extract of cannabis and a further physiologically active agent selected from the group consisting of nutraceuticals, nootropics and psychoactives.

According to a second embodiment described herein, there is provided a method for preparing a composition, the method comprising combining: a first active agent formulated for buccal delivery and a second active agent formulated for gastric delivery, the composition comprising one or more constituent, derivative or extract of cannabis and a further physiologically active agent selected from the group consisting of nutraceuticals, nootropics and psychoactives.

According to a third embodiment provided herein, there is provided an oral product comprising a composition as described above.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a”, “an” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the composition including water. Unless otherwise indicated, reference to “weight percent” of a composition reflects the total wet weight of the composition (i.e., including water).

According to a first embodiment described herein, there is provided a composition comprising a first active agent formulated for buccal delivery and a second active agent formulated for gastric delivery, the composition comprising one or more constituent, derivative or extract of cannabis and a further physiologically active agent. In some embodiments, the further physiologically active agent is selected from the group consisting of nutraceuticals, nootropics and psychoactives.

### Active Agent

The first active agent and the second active agent may be the same or different. The first and second active agents are compounds which have a pharmacological activity or modify physiological functions in human beings.

The first and second active agents may be selected from nutraceuticals, nootropics or psychoactives. The composition further comprises a further physiological active agent which may be selected from the group consisting of nutraceuticals, nootropics or psychoactives. From herein onwards, the first and second active agents and further physiological active agent may be collectively referred to as “active agents” or “agents”.

The particular percentages of active agents present will vary depending upon the desired characteristics of the particular product. Typically, an active agent or combination thereof is present in a total concentration of at least about 0.001% by weight of the composition, such as in a range from about 0.001% to about 20%. In some embodiments, each active agent or alternatively the combination of active agents is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, each active agent or alternatively the combination of active agents is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active agents are provided herein below.

The active agents may be naturally occurring or synthetically obtained. In one embodiment, the active agents may comprise for example nicotine, caffeine, taurine, theine, vitamins such as B6 or B12 or C, melatonin, cannabinoids, gamma-aminobutyric acid (GABA), theanine, a botanical or combinations thereof. Included are also physiological active constituents, derivatives, or extracts thereof of the afore-mentioned.

In some embodiments, the active agents comprise nicotine. In some embodiments, the active agents comprise caffeine, melatonin or vitamin B12.

The active agents may comprise one or more constituents, derivatives or extracts of tobacco, cannabis or another botanical. In one embodiment, the active agents may comprise one or more constituents, derivatives or extracts of cannabis, such as one or more cannabinoids or terpenes.

The composition of the present invention comprises a cannabinoid and in one embodiment, the first and second active agents comprise a cannabinoid.

### Constituent, Derivative or Extract of Cannabis

As used herein, any compound or mixture of compounds which may be obtained from cannabis may be a constituent, derivative or extract thereof, including synthetic versions of such compound(s) or such compound(s) derived from other natural sources.

In some embodiments the constituent, derivative or extract of cannabis comprises, or is, one or more compounds selected from: cannabinoids (such as phytocannabinoids that may optionally be THC and/or CBD); terpenes (such as triterpenes); alkaloids; and flavonoids.

In some embodiments the constituent, derivative or extract of cannabis comprises one or more compounds selected from: cannabinoids (such as phytocannabinoids) and terpenes (such as triterpenes).

In some embodiments the constituent, derivative or extract of cannabis comprises one or more cannabinoids, such as phytocannabinoids.

Cannabinoids are a class of natural or synthetic chemical compounds which act on cannabinoid receptors (i.e., CB1 and CB2) in cells that repress neurotransmitter release in the brain. Cannabinoids may be naturally occurring (phytocannabinoids) from plants such as cannabis, from animals (endocannabinoids), or artificially manufactured (synthetic cannabinoids). Cannabis species express at least 85 different phytocannabinoids, and are divided into subclasses, including cannabigerols, cannabichromenes, cannabidiols, tetrahydrocannabinols, cannabinols and cannabinodiols, and other cannabinoids. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), and tetrahydrocannabivarinic acid (THCV A).

In some embodiments, the cannabinoids are phytocannabinoids.

In some embodiments, the terpenes are triterpenes.

In particular embodiments, the constituent, derivative or extract of cannabis comprises, or is, tetrahydrocannabinol (THC) and/or cannabidiol (CBD).

In some embodiments, the constituent, derivative or extract of cannabis comprises, or is, THC.

In particular embodiments, the constituent, derivative or extract of cannabis comprises, or is, CBD.

In some embodiments, the constituent, derivative or extract of cannabis is present in an amount of from about 0.1 to about 30% by weight, based on the total weight of the composition. In some embodiments, the constituent, derivative or extract of cannabis is present in the composition in an amount of at least about 0.1%, at least about 0.2%, at least about 0.3%, at least about 0.4%, at least about 0.5%, at least about 0.6%, at least about 0.7%, at least about 0.8%, or at least about 0.9%. In some embodiments, the constituent, derivative or extract of cannabis is present in the composition in an amount of no more than about 1%, about 2%, about 3%, about 4%, about 5%, about 6%, about 7%, about 8%, about 9%, about 10%, about 15%, about 20%, or no more than about 30% by weight, based on the total weight of the composition.

### Terpenes

Active agents suitable for use in the present disclosure can also be classified as terpenes, many of which are associated with biological effects, such as calming effects.

Terpenes are understood to have the general formula of (C5H8)n and include monoterpenes, sesquiterpenes, and diterpenes. Terpenes can be acyclic, monocyclic or bicyclic in structure.

Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

### Botanicals

As noted herein, the active agents may comprise or be derived from one or more botanicals or constituents, derivatives or extracts thereof. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). The material may be produced synthetically. As used herein, the term “botanical” includes but is not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition.

Example botanicals are tobacco, eucalyptus, star anise, hemp, cocoa, cannabis, fennel, lemongrass, peppermint, spearmint, rooibos, chamomile, flax, ginger, ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v., Mentha spicata crispa, Mentha cardifolia, Mentha longifolia, Mentha suaveolens variegata, Mentha pulegium, Mentha spicata c.v. and Mentha suaveolens

In some embodiments, the active agents comprise or are derived from one or more botanicals or constituents, derivatives or extracts thereof and the botanical is tobacco.

Reference to botanical material as “non-tobacco” is intended to exclude tobacco materials (i.e., does not include any Nicotiana species). In some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

In some embodiments, the active agents comprise or are derived from one or more botanicals or constituents, derivatives or extracts thereof and the botanical is selected from eucalyptus, star anise, cocoa and hemp.

In some embodiments, the active agents comprise or are derived from one or more botanicals or constituents, derivatives or extracts thereof and the botanical is selected from rooibos and fennel.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the composition.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the composition.

### Stimulants

In some embodiments, the active agent comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active agent comprises caffeine. In some embodiments, the caffeine is present in an encapsulated form. One example of an encapsulated caffeine is Vitashure®, available from Balchem Corp., 52 Sunrise Park Road, New Hampton, NY, 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition. In some embodiments, the composition comprises caffeine in an amount of from about 1.5 to about 6% by weight, based on the total weight of the composition.

Amino acids

In some embodiments, the active agent comprises an amino acid. As used herein, the term “amino acid” refers to an organic compound that contains amine (—NH2) and carboxyl (—COOH) or sulfonic acid (SO3H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “nonproteinogenic” is meant that either the amino acid is not found naturally in protein, or is not directly produced by cellular machinery (e.g., is the product of post-translational modification).

Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active agent comprises theanine. In some embodiments, the active agent comprises GABA. In some embodiments, the active agent comprises a combination of theanine and GABA. In some embodiments, the active agent is a combination of theanine, GABA, and lemon balm. In some embodiments, the active agent is a combination of caffeine, theanine, and ginseng. In some embodiments, the active agent comprises taurine. In some embodiments, the active agent is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

### Vitamins/Minerals

In some embodiments, the active agent comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active agent comprises vitamin C. In some embodiments, the active agent is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the composition.

In some embodiments, the active agent comprises a mineral or combination of minerals. As used herein, the term “mineral” refers to a chemical compound with a defined chemical composition and a specific crystal structure that occurs naturally in pure form. In some embodiments, the active agent comprises a magnesium-based mineral compounds, e.g., such as magnesium gluconate, magnesium citrate, and the like. When present, a mineral or combination of minerals (e.g., magnesium gluconate, magnesium citrate, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the composition.

### Antioxidants

In some embodiments, the active agent comprises one or more antioxidants. As used herein, the term “antioxidant” refers to a substance which prevents or suppresses oxidation by terminating free radical reactions, and may delay or prevent some types of cellular damage.

Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, bacopa monniera, withania somnifera, Lion's mane, and silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12 (2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the composition.

### Nicotine Component

In certain embodiments, the active agent comprises a nicotine component. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine component can be employed in the form of a salt. Salts of nicotine can be provided using the types of agents and techniques set forth in U.S. Pat. No. 2,033,909 to Cox et al. and Perfetti, Beitrage Tabakforschung Int., 12:43-54 (1983), which are incorporated herein by reference.

Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C115HMR, or Doshion P551. See, for example, U.S. Pat. No. 3,901,248 to Lichtneckert et al., which is incorporated herein by reference. Another example is a nicotine polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the composition, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the composition. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the composition.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active agent comprises a nicotine component (e.g., any product or composition of the disclosure, in addition to comprising any active agent or combination of active agents as disclosed herein, may further comprise a nicotine component).

### Pharmaceutical Agents

In some embodiments, the active agent comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl) propanoic acid), phosphatidylserine, myoinositol, docosahexaenoic acid (DHA, Omega-3), arachidonic acid (AA, Omega-6), S-adenosylmethionine (SAM), beta-hydroxy-betamethylbutyrate (HMB), citicoline (cytidine-5′-diphosphate-choline), and cotinine. In some embodiments, the active agent comprises citicoline.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the composition.

In some embodiments, the composition is substantially free of any API. By “substantially free of any API” means that the composition does not contain, and specifically excludes, the presence of any API as defined herein, such as any Food and Drug Administration (FDA) approved therapeutic agent intended to treat any medical condition.

### Formulated for Buccal Delivery

In the composition of the present invention, the first active agent is formulated for buccal delivery. By buccal delivery it is meant that the active agent is formulated to diffuse through the oral mucosa and enter the bloodstream directly. Buccal delivery allows improved bioavailability and a more rapid onset of physiological effect due to the bypass of the digestive system and first pass metabolism. Buccal delivery may encompass sublabial administration in which the active agent is placed between the lip and the gum and sublingual administration in which the active agent is placed under the tongue.

Constituents, derivatives or extracts of cannabis, such as cannabinoids, are highly lipophilic molecules with very low aqueous solubility. Formulation can therefore play a critical role in the solubility and delivery of the active. Such formulation may include salt formation (i.e., pH adjustment), cosolvency (e.g., ethanol, propylene glycol, PEG400, etc.), micellization (e.g., polysorbate 80, cremophor ELP, etc.), (nano)-(micro)-emulsification, complexation (e.g., cyclodextrins), and encapsulation in lipid-based formulations (e.g., liposomes) and nanoparticles.

In one embodiment, the constituent, derivative or extract of cannabis is dissolved, dispersed, solubilised or dissoluted in an aqueous environment. In one embodiment, the composition comprises a means for solubilising the constituent, derivative or extract of cannabis in an aqueous environment. The means for solubilising may comprise a solvent in which the one or more constituent, derivative or extract of cannabis is at least partially dissolved. The weight ratio of solubilising agent to cannabinoid is typically in the range of from 100:1 to 5:1, preferably in the range of from 50:1 to 10:1 and more preferably in the range of from 30:1 to 10:1.

Said aqueous environment may be provided in a carrier such as an emulsion system, for example an oil-in-water emulsion, or the matrix of a microcapsule, microsphere or nanosphere system. The agent may be encapsulated or dispersed on the surface of the microsphere or nanosphere.

An oil-in-water emulsion can be prepared using a combination of a pharmaceutically acceptable oil and emulsifier. The cannabinoid is dissolved in the oil phase which is then mixed with an aqueous phase typically containing a stabiliser under vigorous mixing, milling or homogenisation.

The oil may be selected from vegetable oils such as soybean oil, olive oil, cotton seed oil, peanut oil, sesame oil and castor oil. Vitamin E (tocopherol) can also be used as the oil phase. This material is also an antioxidant and can help to stabilise the chosen constituent, derivative or extract of cannabis which may be prone to oxidation. Pharmaceutically acceptable derivatives of tocopherol are also included, such as the esters of tocopherol, e.g. linoleate, nicotinate, acetate or acid succinate ester.

A further emulsifier is a block copolymer containing a polyoxyethylene block, i.e. a block made up of repeating ethylene oxide moieties. A suitable emulsifier of this type is Poloxamer, i.e. a polyoxyethylene-polyoxypropylene block copolymer, such as Poloxamer 188.

Another emulsifier is a phospholipid emulsifier. This can be any pharmaceutically acceptable material derived from soybeans or eggs, e.g. soy or egg lecithins. Egg lecithins, such as the material provided by Lipoid (Germany) known as Lipoid E80, contains both phosphatidylcholine and phosphatidyl ethanoline. Other phospholipid materials can also be used including phospholipidpolyethylene glycol (PEG) conjugates (PEGylated phospholipids) that have been described for use in liposome systems.

The stability of the emulsion can be enhanced by the addition of a pharmaceutically acceptable co-emulsifier. Suitable co-emulsifiers include the fatty acids and salts thereof and bile acid and salts thereof such as deoxycholic acid. Suitable fatty acids are those having greater than 8 carbon atoms in their structure such as oleic acid. Suitable salts are the pharmaceutically acceptable salts such as the alkali metal, e.g. Na and K, salts. These co-emulsifiers can be added at a concentration of approximately 1% w/v.

The quantity of oil in the emulsion can be from 5 to 50% on a v/v basis, preferably from to 50% v/v and more preferably from 15 to 25% v/v. The constituent, derivative or extract of cannabis is typically dissolved in the oil phase at a concentration of 0.1 to 20% w/v, preferably from 1 to 10% w/v, i.e. from 0.1 to 20, preferably from 1 to 10 g of drug in 100 ml of oil.

In one embodiment, the means for solubilising the one or more constituent, derivative or extract of cannabis in an aqueous environment encapsulates the one or more constituent, derivative or extract of cannabis. The encapsulation may be by a molecular encapsulant, such as a cyclodextrin. Cyclodextrins are cyclic oligosaccharides which comprise glucopyranose units. Cyclodextrin and cyclodextrin derivatives which may be useful in the present invention include α-cyclodextrin, β-cyclodextrin, γ-cyclodextrin, hydroxypropyl-β-cyclodextrin, dimethyl-β-cyclodextrin, sulphobutylether cyclodextrin, 2,6-dimethyl-β-cyclodextrin, 2,3,6-trimethyl-β-cyclodextrin.

In another embodiment, the encapsulation may be by a micelle comprising a surfactant. The surfactant may be selection from the group consisting of long chain triglycerides such as C16-C18 triglycerides. In one embodiment, the surfactant may be linolenic acid.

Examples of suitable surfactants include: glyceryl monooleate; and sodium lauryl sulfate (sodium dodecyl sulfate, SLS, or SDS), docusate sodium, lecithins, polyoxyethylene sorbitan fatty acid esters (Polysorbate, Tween®)), polyoxyethylene 15 hydroxy stearate (Macrogol 15 hydroxy stearate, Solutol HS15®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®), and polyoxyethylene nonylphenol ether (Nonoxynol®) and sugar esters.

In some embodiments, the surfactant is present in an amount of from about 0.5 to about 10% by weight, based on the total weight of the composition.

In one embodiment, the constituent, derivative or extract of cannabis may be encapsulated or dispersed on the surface of a microsphere or nanosphere. PLGA (poly (lactic-co-glycolic acid)) may be used as it is mechanically strong, hydrophobic, biocompatible and degrades into toxicologically acceptable products that are eliminated from the body.

Alternatively, a bioadhesive starch microsphere may be used. Other bioadhesive microspheres that may be used in the present compositions include those made from chitosan, polyvinyl pyrrolidone, alginate, polycarbophil, pectin, hyaluronic acid (and esters thereof), agar agarose, dextran, ovalbumin, collagen, starch, albumin and casein. Said materials can interact with mucus or a mucosal surface and thereby provide retention of the constituent, derivative or extract of cannabis at a mucosal surface for a period of time longer than that found for a simple liquid or powder system. The microspheres can be produced by various processes known to the person skilled in the art such as a spray drying process or an emulsification process.

### Formulated for Gastric Delivery

The compositions provided herein are intended for oral administration, and the intention is for the majority of the second active agent provided in the composition to have their effect by enteral absorption through the intestines. A small proportion of the second active agent may be absorbed through the mucosa in the mouth (e.g., via buccal or sublingual delivery), but most will be swallowed and should be taken up by enterocyte intestinal absorption (i.e., via enteral delivery).

Active agent delivery within the oral cavity can be subject to rapid elimination of the drug due to the flushing action of saliva. It is therefore desirable within the composition of the present invention to also have the active agent formulated for gastric delivery. By gastric delivery it is meant delivery to the gastrointestinal tract.

A common issue associated with the gastric delivery of compositions comprising an active agent is the bioavailability of the active agent. Gastric, or more accurately, enteral delivery generally enjoys the benefits of high intestinal surface area and rich mucosal vasculature to provide good absorption and bioavailability. However, for active agents that are poorly soluble in the aqueous environment of the small and large intestines, it is challenging to present these compound in a form that will allow them to be absorbed across the mucosa. Further, once absorbed, the majority of constituent, derivative or extract of cannabis will be transported via the portal vein to the liver where it will undergo first-pass metabolism and so will not provide the desired physiological effects.

Even when a constituent, derivative or extract of cannabis is provided in a form which has improved aqueous solubility, constituent, derivative or extract of cannabis not absorbed by the oral mucosa may come out of solution in the alimentary canal. When this happens, for example by a process of crystallization or precipitation, the bioavailability of the constituent, derivative or extract of cannabis is significantly decreased.

A “spring” is defined as a high energy species that drives supersaturation of an API, in this case the constituent, derivative or extract of cannabis. Such a high energy species is less stable and, therefore, more soluble than an analogous relatively more stable form (e.g., free form, polymorph, etc.). The intrinsic solubility of a high energy species can be 1.5, 2, 3, 4, 5, 6, 7, 8, 9, 10, 25, 50, 75, 100 or more times greater than for an analogous more stable form. As discussed above, one embodiment of the present invention provides for the constituent, derivative or extract of cannabis in a form with improved aqueous solubility. Once dissolution takes place, inhibition of precipitation becomes important. The inhibition of precipitation acts as a “parachute” to slow the rate of the constituent, derivative or extract of cannabis precipitating from solution. Another embodiment of the present invention therefore provides for a constituent, derivative or extract of cannabis in a formulation which inhibits precipitation upon initial dissolution.

The ‘parachute’ can be achieved by precipitation inhibitors. In one embodiment, the composition therefore comprises an additive which slows or inhibits crystallisation of the constituent, derivative or extract of cannabis in an aqueous environment. Polymers such as cellulose derivatives (e.g. hydroxypropyl cellulose-HPC, hydroxypropyl methylcellulose HPMC), vinyl polymers [e.g. polyvinylpyrrolidone-PVP, polyvinylpyrrolidone vinyl-acetate PVP-vinyl acetate (VA)], ethylene polymers (e.g. polyethylene glycol-PEG) and mixtures thereof may be used. In one embodiment the active is selected from PVP and HPC and mixtures thereof. Without wishing to be bound by theory, the mechanisms usually involve increasing the solubility and/or decreasing nucleation and crystal growth, by increasing the viscosity (which results in reduced molecular mobility, thus decreasing nucleation and crystal growth), adsorption onto the crystal surface (which hinders crystal growth), or changing the level of solvation at the crystal/liquid interface (which slows the incorporation of drug molecules into a crystal lattice).

In one embodiment, the active agent formulated for gastric delivery may optionally be coated with one or more materials suitable for the regulation of release or for the protection of the formulation. In one embodiment, coatings are provided to permit either pH-dependent or pH-independent release, e.g., when exposed to gastrointestinal fluid. A pH-dependent coating serves to release the cannabinoid in desired areas of the gastro-intestinal (GI) tract, e.g., the stomach or small intestine. Coatings which are pH-dependent and may be used in accordance with the present invention include shellac, cellulose acetate phthalate (CAP), polyvinyl acetate phthalate (PVAP), hydroxypropylmethylcellulose phthalate, and methacrylic acid ester copolymers, zein, and the like. The coating may be applied in the form of an organic or aqueous solution or dispersion.

### Enhancing Intestinal Absorption

Orally administered active substances typically show low bioavailability due to their degradation by enzymes in the gastrointestinal tract, and due to the difficulty of absorbing them in the small intestine. Additives may be included in the compositions to promote enteral absorption.

For example, medium chain fatty acid salts may enhance enteral delivery of the constituent, derivative or extract of cannabis by increasing paracellular permeability of the intestinal epithelium.

Liposomes are vesicles formed from lipid bilayers made up of one or more types or lipid. The bilayer provides a hydrophobic environment whilst there can be an aqueous phase inside the vesicle. Hydrophilic molecules can be loaded into the interior of liposomes, whilst hydrophobic or lipophilic molecules are incorporated into the lipid bilayer of the liposome.

Liposomes have the ability to encapsulate and protect active substances and to increase their absorption into enterocytes. Liposomes can protect labile active substances from denaturation by the harsh conditions in the gastrointestinal tract. The lipids of liposomes can also be utilized to stimulate the production of chylomicrons in enterocytes, thus enhancing drug transport into the lymphatic system. Furthermore, enterocyte uptake of liposomes can be controlled with their size; smaller showed higher uptake.

Suitable liposome-forming lipids include, for example, phospholipids such as phosphatidylcholine. Other lipids may also be used.

### Increasing Intestinal Lymphatic Transport

Following oral administration and enteral absorption, the second active agent will either enter the portal vein or intestinal lymphatic system. The main factors that control the route that they take are molecular mass and solubility. It has been found that some active agents, including many constituents, derivatives or extracts of cannabis, are preferentially transported via the portal vein. There, they immediately accumulate in the liver and are then metabolized by enzymes, which lowers their concentration in the bloodstream.

The alternative route for delivering active substances to the systemic circulation is the intestinal lymphatic pathway. The intestinal lymphatic pathway can bypass first-pass metabolism in the liver, thus increasing bioavailability. It is therefore desirable to increase intestinal lymphatic transport of the second active agent.

As mentioned above, liposomes may not only enhance enteral delivery but also enhance transport to the lymphatic system.

Co-administration of lipids generally may also enhance transport of the second active agent to the lymphatic system. Therefore, in some embodiments, the composition comprises a lipid component. The lipids may include, for example, phospholipids, long-chain triglycerides and fatty acids such as oleic acid.

In some embodiments, the composition comprises an additive that acts as a metabolism-directing agent, enhancing transport to the lymphatic system. Suitable metabolism directing agents include terpenes, a grapefruit extract, piperine which is typically extracted from black pepper, or combinations thereof. The metabolism-directing agent advantageously directs the second active to the gastric system. This may alter the time for the second active agent to take effect. In addition, the particle size also has an effect of the metabolism of the second active agent, and this may be selected advantageously to have a synergistic effect with the metabolism directing agent.

Some terpenes provide an entourage effect when used in combination with constituents, derivatives or extracts of cannabis or cannabimimetics.

In some embodiments, the metabolism-directing agent is a terpene. Terpenes are understood to have the general formula of (C₅H₈) n and include monoterpenes, sesquiterpenes, and diterpenes. Terpenes can be acyclic, monocyclic or bicyclic in structure. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

An advantage of the inclusion of terpenes is that many terpenes are associated with biological effects, such as calming effects. This can provide the user with an additional effect, on top of that which is provided with the constituent, derivative or extract of cannabis. The rate of metabolism of the terpene may also be different to the constituent, derivative or extract of cannabis, so as to provide the user with an effect at different times.

### Flavour

In some embodiments, the composition further comprises a flavour. As used herein, a “flavouring agent,” “flavour” or “flavourant” is any flavourful or aromatic substance capable of altering the sensory characteristics associated with the oral product.

Examples of sensory characteristics that can be modified by the flavouring agent include taste, mouthfeel, moistness, coolness/heat, and/or fragrance/aroma. Flavouring agents may be natural or synthetic, and the character of the flavours imparted thereby may be described, without limitation, as fresh, sweet, herbal, confectionary, floral, fruity, or spicy. Specific types of flavours include, but are not limited to, vanilla, coffee, chocolate/cocoa, cream, mint, spearmint, menthol, peppermint, wintergreen, eucalyptus, lavender, cardamom, nutmeg, cinnamon, clove, cascarilla, sandalwood, honey, jasmine, ginger, anise, sage, licorice, lemon, orange, apple, peach, lime, cherry, strawberry, trigeminal sensates, terpenes, and any combinations thereof. See also, Leffingwell et al., Tobacco Flavoring for Smoking Products, R. J. Reynolds Tobacco Company (1972), which is incorporated herein by reference. Flavouring agents also may include components that are considered moistening, cooling or smoothening agents, such as eucalyptus. These flavours may be provided neat (i.e., alone) or in a composite, and may be employed as concentrates or flavour packages (e.g., spearmint and menthol, orange and cinnamon, lime, pineapple, and the like).

Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavouring agent may be provided in a spray-dried form or a liquid form.

The flavour may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavour comprises menthol, spearmint and/or peppermint. In some embodiments, the flavour comprises flavour components of cucumber, blueberry, citrus fruits and/or redberry. In some embodiments, the flavour comprises eugenol. In some embodiments, the flavour comprises flavour components extracted from tobacco. In some embodiments, the flavour comprises flavour components extracted from cannabis.

In some embodiments, the composition may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing warming/heating, cooling, tingling, numbing effect. A suitable warming effect agent may be, but is not limited to, vanillyl ethyl ether or capsaicin and a suitable cooling agent may be, but is not limited to eucolyptol or WS-3.

The amount of flavour or sensate utilized in the composition can vary, but is typically up to about 10% by weight, and certain embodiments are characterized by a flavoring or sensate agent content of at least about 0.1% by weight, such as about 0.5 to about 10%, about 1 to about 5%, or about 2 to about 4% weight, based on the total weight of the composition.

In order to improve the organoleptic properties of a composition as disclosed herein, the composition may include one or more taste modifying agents (“taste modifiers”) which may serve to mask, alter, block, or improve e.g., the flavour of a composition as described herein. Non-limiting examples of such taste modifiers include analgesic or anaesthetic herbs, spices, and flavours which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the composition may comprise a cannabinoid or other component having a bitter taste, and a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., a cannabinoid). Suitable taste modifiers include, but are not limited to, capsaicin, gamma-amino butyric acid (GABA), adenosine monophosphate (AMP), lactisole, or a combination thereof.

When present, a representative amount of taste modifier is about 0.01% by weight or more, about 0.1% by weight or more, or about 1.0% by weight or more, but will typically make up less than about 10% by weight of the total weight of the composition, (e.g., from about 0.01%, about 0.05%, about 0.1%, or about 0.5%, to about 1%, about 5%, or about 10% by weight of the total weight of the composition).

### Functional Materials

The composition may further comprise one or more other functional materials which may be selected from one or more of buffering agents, fillers, binders, organic acids, salts, humectants, colorants, sweeteners, taste modifiers, emulsifier, processing aid, oral care additive, preservatives/stabilisers, water and combinations thereof.

### Buffering Agents

In certain embodiments, the composition of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Non-limiting examples of suitable buffers include alkali metals acetates, glycinates, phosphates, glycerophosphates, citrates, carbonates, hydrogen carbonates, borates, or mixtures thereof. In some embodiments, the buffer is sodium bicarbonate.

Where present, the buffering agent is typically present in an amount less than about 5% by weight, based on the weight of the composition, for example, from about 0.1% to about 5%, such as, e.g., from about 0.1% to about 1%, or from about 0.1% to about 0.5% by weight, based on the total weight of the composition.

### Fillers

The compositions as described herein comprise one or more fillers. Fillers may fulfil multiple functions, such as enhancing certain organoleptic properties such as texture and mouthfeel, enhancing cohesiveness or compressibility of the product, and the like.

The amount of filler can vary, but is typically greater than about 20%, and up to about 75% of the composition by weight, based on the total weight of the composition. A typical range of filler within the composition can be from about 20 to about 75% by total weight of the composition, for example, from about 20, about 25, or about 30, to about 35, about 40, about 45, or about 50% by weight (e.g., about 20 to about 50%, or about 25 to about 45% by weight). In certain embodiments, the amount of filler is at least about 20% by weight, such as at least about 25%, or at least about 30%, or at least about 35%, or at least about 40%, based on the total weight of the composition. In some embodiments, the amount of filler within the composition can be from about 38 to about 58% by total weight of the composition.

Generally, fillers are porous particulate materials and are cellulose-based. For example, suitable fillers are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof.

Non-limiting examples of derivatives of non-tobacco plant material include starches (e.g., from potato, wheat, rice, corn), natural cellulose, and modified cellulosic materials. Additional examples of potential fillers include maltodextrin, dextrose, calcium carbonate, calcium phosphate, lactose, mannitol, xylitol, and sorbitol. Combinations of fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the composition based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

Additional examples of potential fillers include maltodextrin, dextrose, calcium carbonate, calcium phosphate, lactose, and sugar alcohols. Combinations of fillers can also be used. In some embodiments, the filler comprises or is a mixture of glucose and starch-derived polysaccharides. One such suitable mixture of glucose and starch-derived polysaccharides is EMDEX®, available from JRS PHARMA LP, USA, 2981 Route 22, Patterson, NY 12563-2359.

In some embodiments, the filler comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). Isomalt is an equimolar mixture of two disaccharides, each composed of two sugars as follows: glucose and mannitol (α-D-glucopyranosido-1,6-mannitol); and glucose and sorbitol (α-D-glucopyranosido-1,6-sorbitol). In some embodiments, the one or more sugar alcohols comprise isomalt. In some embodiments, the one or more sugar alcohols is isomalt.

In some embodiments, the filler comprises a combination of isomalt and EMDEX®. In some embodiments, the one or more sugar alcohols is a combination of isomalt and EMDEX®.

In some embodiments, the one or more sugar alcohols is a combination of two or even three sugar alcohols. In some embodiments, the combination of sugar alcohols comprises or is isomalt and maltitol.

The total amount of sugar alcohols can vary, but is typically greater than about 30%, and up to about 95% of the composition by weight, based on the total weight of the composition. A typical range of sugar alcohols within the composition can be for example, from about 35, about 40, about 45, about 50, or about 55, to about 60, about 65, about 70, about 75, about 80, about 85, about 90, or about 95%, by weight. In certain embodiments, the amount of sugar alcohol is at least about 50% by weight, such as is at least about 55% by weight, or at least about 60%, or at least about 65%, or at least about 70%, or at least about 75%, or at least about 80%, or at least about 85%, based on the total weight of the composition.

In particular embodiments, the sugar alcohol is isomalt in an amount of from about 35 to about 55% by weight, based on the total weight of the composition, such as from about 35, about 40, or about 45, to about 50 or about 55% by weight.

In particular embodiments, the sugar alcohol is a combination of isomalt in an amount of from about 10 to about 25% by weight, such as about 10, about 15, about 20, or about 25% by weight; and maltitol in an amount of from about 50 to about 75% by weight, such as about 50, about 55, about 60, about 65%, about 70, about 75% by weight.

In particular embodiments, the filler is a combination of isomalt in an amount of from about 30 to about 50% by weight, based on the total weight of the composition, such as about 30, about 35, about 40, about 45, or about 50% by weight; and a glucose-polysaccharide blend (e.g., EMDEX®) in an amount of from about 35 to about 55% by weight, based on the total weight of the composition, such as about 35, about 40, about 45, or about 50% by weight.

### Binders

A binder (or combination of binders) may be employed in certain embodiments, in amounts sufficient to provide the desired physical attributes and physical integrity to the composition, and binders also often function as thickening or gelling agents. Typical binders can be organic or inorganic, or a combination thereof. Representative binders include cellulose derivatives (e.g., cellulose ethers), povidone, sodium alginate, starch-based binders, pectin, gums, carrageenan, pullulan, zein, and the like, and combinations thereof. In some embodiments, the binder comprises pectin or carrageenan or combinations thereof.

The amount of binder utilized in the composition can vary based on the binder and the desired composition properties, but is typically up to about 30% by weight, and certain embodiments are characterized by a binder content of at least about 0.1% by weight, such as about 0.5 to about 30% by weight, or about 1 to about 10% by weight, based on the total weight of the composition.

In certain embodiments, the binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the composition.

In some embodiments, the binder comprises pectin. Pectins are natural polymers related to carbohydrates and which are acidic heteropolysaccharides (polysaccharides comprising multiple monosaccharide units). As opposed to carbohydrates, the pectin C-6 position contains a carboxylic acid (or corresponding methyl ester or carboxamide) group instead of a hydroxymethyl group. The principal subunit is known as galacturonic acid, which can be copolymerized with L-rhamnose. Other sugars are featured as side-chain substituents. Pectin acts as a thickening and gelling agent. Pectin isolated from sources such as apple pomace, citrus peels, sugarbeet waste from sugar manufacturing, sunflower heads discarded from seed harvesting, mango waste, and other commercially available pectins may be used. In combination with certain sugars, under acidic conditions (e.g., a pH of from about 2.5 to about 5), or in the presence of a gelation agent (calcium or other divalent alkaline earth elements), pectins may provide a gel or gum consistency to compositions as disclosed herein. In some embodiments, the binder comprises low methoxy pectin. Suitable low methoxy pectins include, for example, “GENU® pectin type LM-104 AS”, available from CP Kelco, Atlanta, GA, USA.

In some embodiments, the binder comprises low methoxy pectin in combination with a gelation agent. In some embodiments, the gelation agent comprises calcium ions, such as, but not limited to, calcium diphosphate. In some embodiments, the binder comprises a high methoxy pectin in combination with an organic acid, described herein below. In some embodiments, the binder comprises a high methoxy pectin in combination with citric acid.

When present, a pectin binder is typically present in an amount of up to about 3% by weight, for example, from about 0.1, about 0.2, about 0.3, about 0.4, about 0.5, about 0.6, about 0.7, about 0.8, about 0.9, or about 1, to about 1.1, about 1.2, about 1.3, about 1.4, about 1.5, about 1.6, about 1.7, about 1.8, about 1.9, about 2, about 2.1, about 2.2, about 2.3. about 2.4, about 2.5, about 2.6, about 2.7, about 2.8, about 2.9, or about 3% by weight, based on the total weight of the composition.

### Organic Acid

In some embodiments, the composition comprises an organic acid. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO2H) or sulfonic acids (—SO2OH). As used herein, reference to organic acid means an organic acid that is intentionally added. In this regard, an organic acid may be intentionally added as a specific mixture ingredient as opposed to merely being inherently present as a component of another mixture ingredient (e.g., the small amount of organic acid which may inherently be present in a mixture ingredient such as a tobacco material). In some embodiments, the one or more organic acids are added neat (i.e., in their free acid, native solid or liquid form) or as a solution in, e.g., water. In some embodiments, the one or more organic acids are added in the form of a salt, as described herein below.

Suitable organic acids will typically have a range of lipophilicities (i.e., a polarity giving an appropriate balance of water and organic solubility). Lipophilicity is conveniently measured in terms of logP, the partition coefficient of a molecule between an aqueous and lipophilic phase, usually water and octanol, respectively. Typically, lipophilicities of organic acids may be between about −2 and about 6.5. In some embodiments, the organic acid may be more soluble in water than in octanol (i.e., having a negative logP value, such as from about −2 to about −1). In some embodiments, the organic acid may be about equally soluble in octanol than in water (i.e., having a logP value of about 0). In some embodiments, the organic acid may be more soluble in octanol than in water (i.e., having a positive logP value, such as from about 1 to about 6.5). In some embodiments, the organic acid has a logP value of from about 1.5 to about 5.0, e.g., from about 1.5, about 2.0, about 2.5, or about 3.0, to about 3.5, about 4.0, about 4.5, or about 5.0.

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C1-C20). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp3 carbon atoms), or may be unsaturated (i.e., having at least one site of unsaturation). As used herein, the term “unsaturated” refers to the presence of a carbon-carbon, sp2 double bond in one or more positions within the alkyl group.

Unsaturated alkyl groups may be mono-or polyunsaturated. Representative straight chain alkyl groups include, but are not limited to, methyl, ethyl, n-propyl, n-butyl, n-pentyl, and n-hexyl.

Branched chain alkyl groups include, but are not limited to, isopropyl, sec-butyl, isobutyl, tertbutyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono-or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo [4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

Heteroaryl and heterocycloalkyl groups can be unsubstituted or substituted. “Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH3, NH2, —NHCH3, —N(CH3)2, —CN, —NC (═O)CH3, —C(═O)—, —C(═O)NH2, and —C(═O)N(CH3)2. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, octanoic acid, nonanoic acid, decanoic acid, undecanoic acid, dodecanoic acid, stearic acid, oleic acid, linoleic acid, linolenic acid, and the like. In some embodiments, the organic acid is an alkyl sulfonic acid.

Non-limiting examples of alkyl sulfonic acids include propanesulfonic acid and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Nonlimiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like. In some embodiments, the organic acid is citric acid, sodium citrate, calcium citrate, or a combination thereof.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Additional non-limiting examples of suitable organic acids include 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), camphoric acid (+), camphor-10-sulfonic acid (+), capric acid, caproic acid, caprylic acid, cinnamic acid, cyclamic acid, decanoic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glycerophosphoric acid, glycolic acid, hippuric acid, isobutyric acid, lactobionic acid, lauric acid, malonic acid, mandelic acid, methanesulfonic acid, naphthalene-1,5-disulfonic acid, naphthalene-2-sulfonic acid, oleic acid, palmitic acid, pamoic acid, pyroglutamic acid, sebacic acid, stearic acid, and undecylenic acid.

In some embodiments, the one or more organic acids is a single organic acid. In some embodiments, the one or more organic acids is a combination of several acids, such as two, three, or more organic acids.

The amount of organic acid present in the composition may vary. Generally, the mixture comprises from about 0.1 to about 10% by weight of organic acid, present as one or more organic acids, based on the total weight of the composition. In some embodiments, the composition comprises about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% organic acid by weight, based on the total weight of the composition. In some embodiments, the composition comprises from about 0.1 to about 0.5% by weight of organic acid, for example, about 0.1, about 0.15, about 0.2, about 0.25, about 0.3, about 0.35, about 0.4, about 0.45, or about 0.5% by weight, based on the total weight of the composition. In some embodiments, the composition comprises from about 0.25 to about 0.35% by weight of organic acid, for example, from about 0.25, about 0.26, about 0.27, about 0.28, about 0.29, or about 0.3, to about 0.31, about 0.32, about 0.33, about 0.34, or about 0.35% by weight, based on the total weight of the composition. In the case where a salt of an organic acid is added (e.g., sodium citrate), the percent by weight is calculated based on the weight of the free acid, not including any counter-ion which may be present.

Organic acids (e.g., citric acid) may be added neat (i.e., as a solid) or in solution, for example, in water. In some embodiments, the organic acid is added as a 50% aqueous solution.

### Salts

In some embodiments, the composition comprises a salt (e.g., an alkali metal salt), typically employed in an amount sufficient to provide desired sensory attributes to the composition. Non-limiting examples of suitable salts include sodium chloride, potassium chloride, ammonium chloride, flour salt, sodium acetate, sodium citrate, calcium citrate, and the like. In some embodiments, the salt is sodium chloride, ammonium chloride, or a combination thereof. In some embodiments, the salt is trisodium citrate, calcium citrate, or a combination thereof.

When present, a representative amount of salt is about 0.1% by weight or more, about 0.5% by weight or more, about 1.0% by weight or more, or about 1.5% by weight or more, but will typically make up about 10% or less of the total weight of the composition, or about 7.5% or less, or about 5% or less (e.g., from about 0.1 to about 5% by weight or from about 0.5 to about 1.5%).

### Humectants

In certain embodiments, one or more humectants may be employed in the composition. Examples of humectants include, but are not limited to, glycerin, propylene glycol, and the like.

Where included, the humectant is typically provided in an amount sufficient to provide desired moisture attributes to the composition. Further, in some instances, the humectant may impart desirable flow characteristics to the composition for depositing in a mold. When present, a humectant will typically make up about 5% or less of the weight of the composition (e.g., from about 0.1 to about 5% by weight), for example, from about 0.1% to about 1% by weight, or about 1% to about 5% by weight, based on the total weight of the composition.

### Colorants

A colorant may be employed in amounts sufficient to provide the desired physical attributes to the composition. Examples of colorants include various dyes and pigments, such as caramel coloring and titanium dioxide. Natural colorants such as curcumin, beet juice extract, spirulina; also a variety of synthetic pigments may also be used. The amount of colorant utilized in the composition can vary, but when present is typically up to about 3% by weight, such as from about 0.1%, about 0.5%, or about 1%, to about 3% by weight, based on the total weight of the composition.

### Sweeteners

In order to improve the sensory properties of the composition according to the disclosure, one or more sweeteners may be added. The sweeteners can be any sweetener or combination of sweeteners, in natural or artificial form, or as a combination of natural and artificial sweeteners.

Examples of natural sweeteners include fructose, sucrose, glucose, maltose, mannose, galactose, lactose, isomaltulose, stevia, honey, and the like. Examples of artificial sweeteners include sucralose, maltodextrin, saccharin, aspartame, acesulfame K, neotame, and the like. In some embodiments, the sweetener comprises one or more sugar alcohols. Sugar alcohols are polyols derived from monosaccharides or disaccharides that have a partially or fully hydrogenated form.

Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, the sweetener is sucralose, acesulfame K, or a combination thereof.

When present, a sweetener or combination of sweeteners may make up from about 0.01 to about 20% or more of the of the composition by weight, for example, from about 0.01 to about 0.1, from about 0.1 to about 1%, from about 1 to about 5%, from about 5 to about 10%, or from about 10 to about 20% by weight, based on the total weight of the composition. In some embodiments, a combination of sweeteners is present at a concentration of from about 0.01% to about 0.1% by weight of the composition, such as about 0.01, about 0.02, about 0.03, about 0.04, about 0.05, about 0.06, about 0.07, about 0.08, about 0.09, or about 0.1% by weight of the composition. In some embodiments, a combination of sweeteners is present at a concentration of from about 0.05% to about 0.5% by weight of the composition, such as about 0.1, about 0.2, about 0.3, about 0.4, or about 0.5% by weight of the composition. In some embodiments, a combination of sweeteners is present at a concentration of from about 1% to about 3% by weight of the composition.

### Taste Modifiers

In order to improve the organoleptic properties of a composition as disclosed herein, the composition may include one or more taste modifying agents (“taste modifiers”) which may serve to mask, alter, block, or improve e.g., the flavor of a composition as described herein. Nonlimiting examples of such taste modifiers include analgesic or anesthetic herbs, spices, and flavors which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. The composition comprises a cannabinoid which may have a bitter taste, and therefore, a taste modifier which masks or blocks the perception of the bitter taste may be advantageous. In some embodiments, the taste modifier is a substance which targets pain receptors (e.g., vanilloid receptors) in the user's mouth to mask e.g., a bitter taste of another component (e.g., a cannabinoid). Suitable taste modifiers include, but are not limited to, capsaicin, gamma-amino butyric acid (GABA), adenosine monophosphate (AMP), lactisole, or a combination thereof.

When present, a representative amount of taste modifier is about 0.01% by weight or more, about 0.1% by weight or more, or about 1.0% by weight or more, but will typically make up less than about 10% by weight of the total weight of the composition, (e.g., from about 0.01%, about 0.05%, about 0.1%, or about 0.5%, to about 1%, about 5%, or about 10% by weight of the total weight of the composition).

### Emulsifier

In certain embodiments, an emulsifier may be added. In some embodiments, the emulsifier is lecithin. For example, lecithin (e.g., soy lecithin or sunflower lecithin) may be added to the composition to provide smoother textural properties to the composition and to improve flowability and mixing of e.g., a lipid with the remaining components of the composition. Emulsifiers (e.g., lecithin) can be used in an amount of about 0.01 to about 5% by dry weight of the composition, such as from about 0.1 to about 2.5%, or from about 0.5 to about 1.5% based on the total weight of the composition.

### Processing Aids

If necessary for downstream processing of the composition, such as granulation, mixing, or molding, a flow aid can also be added to the composition in order to enhance flowability of the composition. In some embodiments, the composition (e.g., melt and chew forms) may be surface treated with anti-stick agents, such as oils, silicones, and the like. Exemplary flow aids include microcrystalline cellulose, silica, polyethylene glycol, stearic acid, calcium stearate, magnesium stearate, zinc stearate, sodium stearyl fumarate, canauba wax, and combinations thereof. In some embodiments, the flow aid is sodium stearyl fumarate.

When present, a representative amount of flow aid may make up at least about 0.5 percent or at least about 1 percent, of the total dry weight of the composition. Preferably, the amount of flow aid within the composition will not exceed about 5 percent, and frequently will not exceed about 3 percent, of the total dry weight of the composition.

### Oral Care Additives

In some embodiments, the composition comprises an oral care ingredient (or mixture of such ingredients). Oral care ingredients provide the ability to inhibit tooth decay or loss, inhibit gum disease, relieve mouth pain, whiten teeth, or otherwise inhibit tooth staining, elicit salivary stimulation, inhibit breath malodor, freshen breath, or the like. For example, effective amounts of ingredients such as thyme oil, eucalyptus oil and zinc (e.g., such as the ingredients of formulations commercially available as ZYTEX® from Discus Dental) can be incorporated into the composition. Other examples of ingredients that can be incorporated in desired effective amounts within the present composition can include those that are incorporated within the types of oral care compositions set forth in Takahashi et al., Oral Microbiology and Immunology, 19 (1), 61-64 (2004); U.S. Pat. No. 6,083,527 to Thistle; and US Pat. Appl. Pub. Nos. 2006/0210488 to Jakubowski and 2006/02228308 to Cummins et al. Other exemplary ingredients of tobacco containing-formulation include those contained in formulations marketed as MALTISORB® by Roquette and DENTIZYME® by NatraRx. When present, a representative amount of oral care additive is at least about 1%, often at least about 3%, and frequently at least about 5% of the total dry weight of the composition. The amount of oral care additive within the composition will not typically exceed about 30%, often will not exceed about 25%, and frequently will not exceed about 20%, of the total dry weight of the composition.

### Other Additives

Other additives can be included in the disclosed composition. For example, the composition can be processed, blended, formulated, combined, and/or mixed with other materials or ingredients. The additives can be artificial, or can be obtained or derived from herbal or biological sources. Examples of further types of additives include thickening or gelling agents (e.g., fish gelatin), emulsifiers, preservatives (e.g., potassium sorbate and the like), disintegration aids, zinc or magnesium salts selected to be relatively water soluble for compositions with greater water solubility (e.g., magnesium or zinc gluconate) or selected to be relatively water insoluble for compositions with reduced water solubility (e.g., magnesium or zinc oxide), or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final composition, with an example range of up to about 10% by weight, based on total weight of the composition (e.g., about 0.1 to about 5% by weight).

The aforementioned additives can be employed together (e.g., as additive formulations) or separately (e.g., individual additive components can be added at different stages involved in the preparation of the final composition). Furthermore, the aforementioned types of additives may be encapsulated as provided in the final product or composition. Exemplary encapsulated additives are described, for example, in WO2010/132444 to Atchley, which has been previously incorporated by reference herein.

### Water

The moisture content (e.g., water content) of the composition, prior to use by a consumer of the product, may vary according to the desired properties. Typically, the composition, prior to insertion into the mouth of the user, is less than about 10% by weight of water, and generally is from about 0.01 to about 10% by weight of water, for example, from about 0.1 to about 1.0% by weight. In some embodiments, there may be no water intentionally added to the composition other than the amount present in various components of the composition.

### Formulation

The composition of the invention is ideally formulated for oral use. The term “formulated for oral use” as used herein means that the composition is provided in a form such that during use, components of the composition (e.g. cannabinoids) pass into the mouth of the user. In certain embodiments, a first active agent, for example a constituent, derivative or extract of cannabis, is formulated for buccal delivery, i.e. through mucous membranes in the user's mouth. In certain embodiments, a second active agent, for example a constituent, derivative or extract of cannabis, is formulated for gastric delivery, e.g. through the user's digestive system. Therefore, in some embodiments, a constituent, derivative or extract of cannabis can be absorbed through the mucous membranes in the mouth and absorbed through the digestive tract when the product is used.

The compositions disclosed herein can be formulated in the form of a solid unit dosage form, a powder or granules. In one embodiment, the particles of said formulation may have a volume mean diameter of 100 to 2000 μm.

A further aspect of the invention relates to a method for preparing a composition, the method comprising combining: a first active agent formulated for buccal delivery and a second active agent formulated for gastric delivery, the composition comprising one or more constituent, derivative or extract of cannabis and a further physiologically active agent selected from the group consisting of nutraceuticals, nootropics and psychoactives. In one embodiment, the one or more constituent, derivative or extract of cannabis is combined with a means for solubilising the one or more constituent, derivative or extract of cannabis in an aqueous environment, and optionally an additive which slows or inhibits crystallisation of the one or more constituent, derivative or extract of cannabis in an aqueous environment.

The manner by which the various components of the composition (e.g., filler, cannabinoid, and the like) are combined may vary. As such, the overall composition with e.g., powdered composition components may be relatively uniform in nature (e.g., homogenous). The components noted above, which may be in liquid or dry solid form, can be admixed in a pretreatment step prior to mixture with any remaining components of the composition, or simply mixed together with all other liquid or dry ingredients. The compositions of the disclosure may be prepared, for example, by dry-blending dry ingredients, such as filler, sweeteners, salts, and the like. In certain embodiments, water can be added to the dry blend at this stage. Additionally, it is optional to add, such as by spraying, cannabinoids and/or flavoring agents to the dry blend, followed by mixing. As such, the composition of the present invention may be spray dried or extruded.

The various components of the composition may be contacted, combined, or mixed together using any mixing technique or equipment known in the art. Any mixing method that brings the composition ingredients into intimate contact can be used, such as a mixing apparatus featuring an impeller or other structure capable of agitation.

Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Ploughshare® types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference. In some embodiments, the components forming the composition are prepared such that the mixture thereof may be used in a starch moulding process for forming the composition. Manners and methods for formulating compositions will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. Nos. 4,725,440 to Ridgway et al., and U.S. Pat. No. 6,077,524 to Bolder et al., each of which is incorporated herein by reference.

In a further aspect of the invention, the composition of the present invention is in the form of an oral product. The oral product may comprise a pouch containing the composition. The pouch may be made of a flexible liquid permeable material. The pouch may be placed next to the soft tissue of the mouth and generate a rapid release of the active when in contact with saliva. Alternatively, the oral product may comprise a dissolving strip comprising the composition. Such strips are described for example, in U.S. Pat. No. 20,160,51510. In one embodiment, the strip may comprise a bioadhesive to adhere the strip to the mucosa. Such an adhesive may be selected from carboxymethylcellulose, polyvinyl alcohol, polyvinyl pyrrolidone (povidone), sodium alginate, methyl cellulose, hydroxyl propyl cellulose, hydroxypropylmethyl cellulose, polyethylene glycols, carbopol, polycarbophil, carboxyvinyl copolymers, propylene glycol alginate, alginic acid, methyl methacrylate copolymers, tragacanth gum, guar gum, karaya gum, ethylene vinyl acetate, dimenthylpolysiloxanes, polyoxyalkylene block copolymers, pectin, chitosan, carrageenan, xanthan gum, gellan gum, locust bean gum, hydroxyethylmethacrylate copolymers and mixtures thereof.

In alternative embodiments, the oral product may be in a form selected from an edible gel, a liquid beverage, a chew, a pastille or a chewing gum. Such formulations are well known to one skilled in the art.

A skilled person will appreciate that all aspects of the invention, whether they relate to, for example, the composition, the method or oral product are equally applicable to all other aspects of the invention. In particular, aspects of the composition for example, may have been described in greater detail than in other aspects of the invention, for example, the method. However, the skilled person will appreciate where more detailed information has been given for a particular aspect of the invention, this information is generally equally applicable to other aspects of the invention.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A composition comprising a first active agent formulated for buccal delivery and a second active agent formulated for gastric delivery, the composition comprising one or more constituent, derivative or extract of cannabis and a further physiologically active agent selected from the group consisting of nutraceuticals, nootropics and psychoactives.

2. A composition as claimed in claim 1, wherein the first active agent, second active agent and or further physiologically active agent are independently selected from the group consisting of constituents, derivatives or extracts of cannabis, nicotine, caffeine, taurine, theine, vitamins such as B6 or B12 or C, melatonin, gamma-aminobutyric acid (GABA), theanine, a botanical or combinations thereof.

3. A composition as claimed in claim 1 or claim 2, wherein the first active agent formulated for buccal delivery comprises one or more constituent, derivative or extract of cannabis.

4. A composition as claimed in any one of claims 1 to 3, wherein the second active agent formulated for gastric delivery comprises one or more constituent, derivative or extract of cannabis.

5. A composition as claimed in any one of claims 1 to 4, wherein the constituent, derivative or extract of cannabis is one or more compounds selected from:
cannabinoids; terpenes; alkaloids; and flavonoids.

6. A composition as claimed in any one of claims 1 to 5, wherein the constituent, derivative or extract of cannabis is selected from the group consisting of: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), and tetrahydrocannabivarinic acid (THCV A).

7. A composition as claimed in any one of claims 1 to 6, wherein the constituent, derivative or extract of cannabis is present in an amount of from about 0.1 to about 30% by weight, based on the total weight of the composition.

8. A composition as claimed in any one of claims 1 to 7, comprising a means for solubilising the one or more constituent, derivative or extract in an aqueous environment.

9. A composition as claimed in claim 8, the means for solubilising the one or more constituent, derivative or extract in an aqueous environment comprising a solvent in which the one or more constituent, derivative or extract is at least partially dissolved.

10. A composition as claimed in claim 8 or claim 9, wherein the means for solubilising the one or more constituent, derivative or extract in an aqueous environment encapsulates the one or more constituent, derivative or extract.

11. A composition as claimed in claim 10, wherein the encapsulation is by a molecular encapsulant, such as a cyclodextrin.

12. A composition as claimed in claim 10, wherein the encapsulation is by a micelle comprising a surfactant.

13. A composition as claimed in any one of claims 1 to 12, comprising a surfactant, optionally wherein the surfactant is selected from the group consisting of: glyceryl monooleate; and sodium lauryl sulfate (sodium dodecyl sulfate, SLS, or SDS), docusate sodium, lecithins, polyoxyethylene sorbitan fatty acid esters (Polysorbate, Tween®), polyoxyethylene 15 hydroxy stearate (Macrogol 15 hydroxy stearate, Solutol HS15®), polyoxyethylene castor oil derivatives (Cremophor® EL, ELP, RH 40), polyoxyethylene stearates (Myrj®), sorbitan fatty acid esters (Span®), polyoxyethylene alkyl ethers (Brij®), polyoxyethylene nonylphenol ether (Nonoxynol®) and sugar esters.

14. A composition as claimed in claim 13, wherein the surfactant is selected from the group consisting of long chain triglycerides (such as C16-C18 triglycerides).

15. A composition as claimed in claim 13 or claim 14, wherein the composition comprises the surfactant in an amount of from about 0.5 to about 10% by weight, based on the total weight of the composition.

16. A composition as claimed in any one of claims 1 to 15, comprising an additive which slows or inhibits crystallisation of the one or more constituent, derivative or extract of cannabis in an aqueous environment.

17. A composition as claimed in claim 16, wherein the additive is selected from the group consisting of polyvinylpyrrolidone (PVP), hydroxypropyl cellulose (HPC) and mixtures thereof.

18. A composition as claimed in any one of claims 1 to 17, comprising one of more components enhancing enterocyte intestinal absorption of the one or more constituent, derivative or extract of cannabis.

19. A composition as claimed in any one of claims 1 to 18, comprising one of more components increasing intestinal lymphatic transport of the one or more constituent, derivative or extract of cannabis.

20. A composition as claimed in claim 19, comprising a terpene, a grapefruit extract or a black pepper extract.

21. A composition as claimed in any one of claims 1 to 20, comprising a flavour or sensate.

22. A composition as claimed in any one of claims 1 to 21, comprising a buffering agent.

23. A composition as claimed in any one of claims 1 to 22, in the form of a solid unit dosage form, a powder or granules.

24. A composition as claimed in claim 23, in the form of particles having a volume mean diameter of 100 to 2000 μm.

25. A method for preparing a composition, the method comprising combining: a first active agent formulated for buccal delivery and a second active agent formulated for gastric delivery, the composition comprising one or more constituent, derivative or extract of cannabis and a further physiologically active agent selected from the group consisting of nutraceuticals, nootropics and psychoactives.

26. A method as claimed in claim 25, wherein the one or more constituent, derivative or extract of cannabis is combined with a means for solubilising the one or more constituent, derivative or extract of cannabis in an aqueous environment, and optionally an additive which slows or inhibits crystallisation of the one or more constituent, derivative or extract of cannabis in an aqueous environment.

27. A method as claimed in claim 25 or claim 26, wherein the composition is spray dried or extruded.

28. An oral product comprising a composition as claimed in any one of claims 1 to 24.

29. An oral product as claimed in claim 28, comprising a pouch containing the composition.

30. An oral product as claimed in claim 28, comprising a dissolving strip comprising the composition.

31. An oral product as claimed in claim 30, the dissolving strip comprising a bioadhesive.

32. An oral product as claimed in claim 28, in a form selected from the group consisting of an edible gel, a liquid beverage, a chew, a pastille or a chewing gum.
